(19) United States
(12) Patent Application Publication
(10) Pub. No.: US 2022/0354319 A1
(43) Pub. Date: Nov. 10, 2022

(54) Toilet Lid Accessory

(71) Applicant: Steve Wood, Paragould, AR (US)

(72) Inventor: Steve Wood, Paragould, AR (US)

(21) Appl. No.: 17/307,097

(22) Filed: May 4, 2021

Related U.S. Application Data
(60) Provisional application No. 63/152,258, filed on Feb. 22, 2021.

(51) Int. Cl. A47K 13/30; A61L 2/10; A61L 2/26

(52) U.S. Cl. CPC A47K 13/302; A61L 2/10; A61L 2/26; A61L 2202/11; A61L 2202/14

(57) ABSTRACT

This present invention relates to a toilet lid mirror removably attached to an interior surface of a toilet lid, which allows the users to check and see if they have thoroughly wiped themselves after urination and/or defecation in toilets. The toiled lid mirror can be conveniently used by all the individuals and is especially helpful for children, people suffering from hemorrhoids, and others to ensure cleanliness and improve personal hygiene. Additionally, the toilet lid mirror provides an easy and quick solution for the users to check and see that they are clean after using the toilets or restrooms.

### CROSS-REFERENCE TO RELATED APPLICATION

The present application claims priority to, and the benefit of, U.S. Provisional Application No. 63/152,258, which was filed on Feb. 22, 2021 and is incorporated herein by reference in its entirety.

### FIELD OF THE INVENTION

The present invention relates generally to the field of mirrors. More specifically, the present invention relates to a removable mirror attached on the underside of a toilet seat lid that allows the users to check and see if they are clean after using a restroom. The toilet lid mirror also assists young children in making sure they have wiped sufficiently, and also affords those individuals that suffer from hemorrhoids the ability to ensure cleanliness. The toilet lid mirror of the present invention offers an easy and quick method for improving personal hygiene. In a further embodiment, one or more LED lights and/or UV sanitization lights may also be included to improve visibility and sanitation. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, billions of individuals use toilets and restrooms on a daily basis around the world for purposes of urination and/or defecation. After using a toilet, some individuals may have difficulty in determining if they have wiped themselves thoroughly after urinating and/or defecating. More specifically, an individual may be unable to view his or her buttocks and genital areas to facilitate proper hygienic cleaning of these areas after urination or defecation. This is a particularly common problem for young children who are learning to use the restroom on their own, and may be unable to determine whether or not their buttocks and genital areas have been properly cleaned after wiping themselves with toilet paper.

Additionally, individuals suffering with hemorrhoids may have difficulty in ensuring cleanliness after defecation. For example, it can be difficult for such individuals to see the underside of their body or the hemorrhoidal areas to check for cleanliness. Unfortunately, an unclean buttocks and/or genital areas may result in soiled clothes, unpleasant odors, infections, rashes, skin irritations and other problems.

While many restrooms include a mirror of some type, most mirrors are typically mounted to a wall above, for example, a sink or in an open space, as opposed to within the privacy of a stall. Therefore, it is not possible for an individual to use these types of mirrors to inspect their private areas, such as the buttocks or genital area, for ensuring cleanliness after urination and/or defecation. Additionally, many individuals may not be comfortable with using a common area mirror for viewing the underside of their body for cleanliness, or may not want to travel from the toilet area to the common area partially unclothed.

Furthermore, while individuals who suffer from hemorrhoids may carry small portable mirrors with them to check their underside for cleanliness, it is oftentimes difficult to both hold and use such small mirrors while checking whether their buttocks or genital areas are clean. Further, children may seek the help of an adult to check if their buttocks and genital areas are thoroughly cleaned post defecation or urination. However, an adult may not always be present, or not may wish to provide such assistance.

Therefore, there exists a long felt need in the art for a toilet lid accessory device that helps individuals to inspect their backside to ensure that they have wiped themselves thoroughly after urination and/or defecation. There is also a long felt need in the art for a toilet lid accessory device that can be easily used by young children to check whether their buttock and/or genital areas are properly cleaned after urination and/or defecation, without seeking the assistance of an adult or another child. Moreover, there is a long felt need in the art for a device which helps individuals suffering from hemorrhoids to quickly check the underside of their body and ensure cleanliness after urination and/or defecation. Further, there is a long felt need in the art for a toilet lid accessory device that serves the additional purposes of both providing additional lighting to the stall, and serving as a disinfecting tool to kill bacteria, germs, pathogens and the like. Finally, there is a long felt need in the art for a toilet lid accessory device that is relatively inexpensive to manufacture, and that is both safe and easy to use.

The subject matter disclosed and claimed herein, in one embodiment thereof, comprises a toilet lid accessory device in the form of a mirror that is designed to be removably attached to an interior surface of a toilet lid. The mirror device comprises a front reflective surface and a rear non-reflective surface, wherein the rear non-reflective surface has an adhesive backing to attach the toilet lid mirror to the interior surface of the toilet lid. The front reflective surface may be used by an individual to inspect his or her underside/backside to maintain personal hygiene, wherein the toilet lid mirror is dimensioned and shaped as per the toilet's lid. The toilet lid mirror may have an LED strip along the mirror edge to provide lighting, and the mirror may have beveled, rounded or flat edges. In a further embodiment, the accessory device may further comprise one or more UV lights to kill bacteria within the toilet bowl when the lid is closed, and a related timer, battery and sensor.

In this manner, the novel toilet lid mirror accessory of the present invention accomplishes all of the forgoing objectives, and provides a relatively safe, easy, convenient and effective solution to assist users in cleaning themselves properly after using the restroom. The toilet lid mirror accessory can be conveniently used by the children for inspecting their underside/backside and for making sure that they have wiped sufficiently. Additionally, the toilet lid mirror accessory is particularly useful for individuals suffering from hemorrhoids, and helps them to ensure cleanliness around the affected area. Finally, the accessory device is also useful for maintaining a sanitary toilet bowl.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a toilet lid mirror designed to be removably attached to an interior surface of a toilet lid. The mirror device is comprised of a front reflective surface and a rear non-reflective surface, wherein the rear non-reflective surface has an adhesive backing for attaching the toilet lid mirror to the interior surface of a toilet lid. The front reflective surface is used by an individual to inspect his or her underside/backside to maintain the individual's personal hygiene, wherein the toilet lid mirror is dimensioned and shaped to coincide with the size and shape of the toilet lid. The toilet lid mirror may further comprise one or more sensors, a battery, a timer, LED lights and UV lights, and wherein the mirror may have beveled, rounded or flat edges.

In a further embodiment of the present invention, a toilet lid system is disclosed. The toilet lid system comprises a toilet lid hingedly connected to a toilet seat, the toilet lid having an interior surface which positions against the toilet seat when covering the toilet seat, a removably attached mirror having a reflective surface and a non-reflective surface wherein the mirror is attached to the interior surface of the toilet lid with a fastening mechanism, and the reflective surface reflects a view of the user's anus, groin and hip areas to maintain personal hygiene and apply medication. The mirror may be of any shape and size that conforms to the shape of the interior surface of the toilet lid.

The mirror can be attached to the toilet lid during manufacturing, or can be an aftermarket accessory that is attached to the toilet lid by the consumer, using an adhesive or other fastening means. For example, in one embodiment, one or more clips placed along the edge of the toilet lid may be used to attach the mirror to the interior surface of the toiled lid. In another embodiment a ring can be used to secure the device to the toilet seat lid. The toilet lid mirror allows individuals to check and see if they are clean after using the bathroom. In this manner, the mirror offers individual users an easy and quick way of improving their personal hygiene. Individuals can also use the device to adjust clothes, apply medication to their backside, etc., which is otherwise difficult to do without a mirror present in the bathroom. The mirror does not decrease the aesthetic appeal of the bathroom, and allows individuals that suffer from hemorrhoids the ability to ensure cleanliness.

In a further embodiment of the present invention, the system may also comprise one or more LED lights for illuminating the area and improving the individual's view of his or her underside/backside. The LED lights or light strip may be powered by, and in electrical communication with, a relatively small disposable or rechargeable battery. Additionally, the battery may also power a sensor, timer and an UV light for a period of time (as determined by the timer) after the sensor has detected that the toilet seat lid has been repositioned into a closed position. In this manner, the UV light can be used to disinfect the interior of the toilet bowl when not in use, and reduce or kill any bacteria, germs, pathogens and the like.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of the reflective and non-reflective surfaces of one potential embodiment of the toilet lid mirror accessory of the present invention designed to be installed along an inner surface of a toilet lid as per the disclosed architecture;

FIG. 2 illustrates a perspective view of one potential embodiment of the toilet lid mirror accessory of the present invention installed along an inner surface of a toilet lid of a toilet as per the disclosed architecture;

FIG. 3 illustrates a sectional view of one potential embodiment of the toilet lid mirror accessory of the present invention installed along an inner surface of a toilet lid as per the disclosed architecture; and

FIG. 4 illustrates a perspective view of one potential embodiment of the toilet lid mirror accessory of the present invention installed along an inner surface of a toilet lid of a toilet as per the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long felt need in the art for a toilet lid accessory device that enables individuals to inspect their underside/backside to ensure that they have cleaned themselves thoroughly after urination and/or defecation. There is also a long felt need in the art for a toilet lid accessory device that can be easily used by young children to check whether their buttock and/or genital areas are properly cleaned after urination and/or defecation, and without seeking the assistance of an adult of another child. Moreover, there is a long felt need in the art for a toilet lid accessory device that helps individuals suffering from hemorrhoids to quickly check the state of their hemorrhoids, and apply medication as necessary. Further, there is a long felt need in the art for a toilet lid accessory device that has an illumination source and that serves the additional purpose of disinfecting toilet surfaces to kill bacteria, germs, pathogens and the like. Finally, there is a long felt need in the art for a toilet lid accessory device that is relatively inexpensive to manufacture, and that is both safe and easy to use.

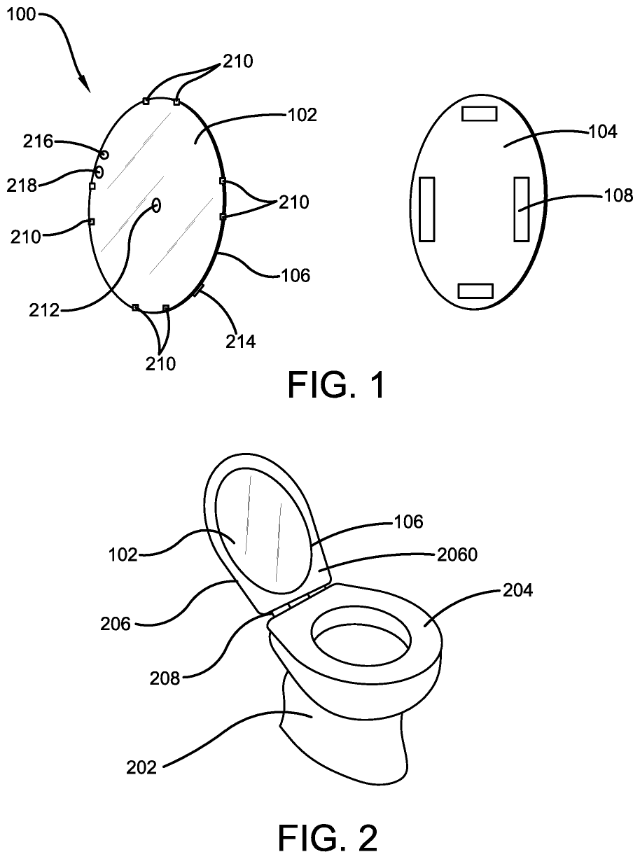

Referring initially to the drawings, FIG. 1 illustrates a perspective view of the reflective and non-reflective surfaces 102, 104 of one potential embodiment of the toilet lid mirror accessory 100 of the present invention designed to be installed along an inner surface 2060 of a toilet lid 206 as per the disclosed architecture. More specifically, the toilet lid mirror accessory or toilet mirror device 100 may be removably attached to the inner surface 2060 of a toilet lid 206, and is sized and shaped to correspond to the size and shape of the inner surface 2060 of the toilet lid 206. For example, in one embodiment, the mirror device 100 is comprised of an oval shape or any other shape that corresponds to the shape of the toilet seat lid 206 (i.e., commonly oval shaped).

The toilet lid mirror accessory 100 is comprised of a reflective surface 102 which faces away from the inner surface 2060 of the toilet lid 206, and is immediately adjacent to the toilet seat 204 when the toilet lid 206 is in the closed position and covers the toilet seat 204. The toilet lid mirror accessory 100 is further comprised of a rear non-reflective surface 104 that is opposite the reflective surface 102 and immediately adjacent to the inner surface 2060 of the toilet lid 206 when the device 100 is properly installed on the toilet lid 206 via a fastening device 108. The fastening device 108 may be any suitable fastening means for removably attaching one object to another such as, but not limited to, one or more releasable adhesive strips, clips, a ring, snaps or the like. For example, the fastening device 108 could be a plurality of clips placed along an outer perimeter of the toilet seat lid 206 and that also attach to the mirror device 100.

The toiled lid mirror device 100 may have beveled or rounded edges 106 to also conform to the shape of the toilet lid, and reduce the likelihood of injury when handling the device 100. The mirror device 100 is secured to the toilet seat lid 206 such that during the repeated use and movement of the toilet seat lid 206, the mirror device 100 remains adhered to the toilet seat lid 206. In one embodiment, the toiled lid mirror 100 may have flat edges to fit and secure to some specific designs of toilet lids.

As previously stated, the mirror device 100 is to ensure cleanliness and maintain personal hygiene after using the toilet 202. The mirror device 100 may also be used to check for hemorrhoids and to apply cream or other ointments to the groin and anal areas. The mirror device 100 can be marketed as an accessory with a new toilet seat 204 or a new toilet lid 206, or can be marketed as an isolated product to be attached to an existing toilet seat 204. The mirror device 100 helps a user in visual identification of hard-to-reach areas such as, but not limited to, the anus, groin or hip area for effective cleanliness and application of medication.

As noted above, in one embodiment, an LED light 210 and/or UV light 212 may also be present along the edge of the mirror device 100 or inner surface 2060 of the toilet lid 206. The LED light 210 can be turned on automatically when the lid 206 is in an open position, via a sensor 216 that detects the positioning of the lid 206. More specifically, the LED light 210 may have a minute motion sensor 216 that automatically turns on the LED light 210 when the toilet seat lid 206 is lifted into the open position to provide adequate illumination for a user for purposes of maintaining personal hygiene and/or to apply medication to the user's backside.

Conversely, the UV light 212 can be turned on automatically when the lid 206 is in a closed position via the sensor 216 that detects the positioning of the lid 206. More specifically, the UV light 210 may have a minute motion sensor 216 that automatically turns on the UV light 212 when the toilet seat lid 206 is lowered into the closed position to disinfect the interior of the toilet 202, the seat 204, the mirror device 100, etc.

Additionally, the mirror device or system 100 may further comprise a battery 214 and a timer 218. The battery may power and be in electrical communication with each of the LED light 210, the UV light 212, the motion sensor 216 and the timer 218. Similarly, the timer 218 may be in electrical communication with at least one LED light 210 and one UV light 212 to turn off power to the same after a user-specified period of time, regardless of the positioning of the toilet seat lid 206, to conserve battery power. Each of the LED light 210 and the UV light 212 would then be reactivated upon the repositioning of the toilet seat lid 206, as detected by the sensor 216.

FIG. 2 illustrates a perspective view of one potential embodiment of the toilet lid mirror accessory 100 of the present invention, installed along an inner surface 2060 of a toilet lid 206 of a toilet 202 as per the disclosed architecture. As shown, the toilet 202 is generally placed on a surface in a private or public bathroom or restroom. The toilet 202 has a toilet seat 204 on which a user generally sits while using the toilet 202. Along with the toilet seat 204, a toilet seat lid 206 is connected through one or more hinges 208 with which the toilet lid 206 covers or uncovers the toilet seat 204. On the inner surface 2060 of the toilet lid 206, the toilet lid mirror 100 is attached with the non-reflective surface adhered to the inner surface 2060, and the reflective surface 102 facing a user, and immediately adjacent to the toilet seat 204 when the lid 206 is lowered into a closed position, and covering the toilet seat 204.

As noted above, the toilet lid mirror device 100 is securely attached to the inner surface 2060 of the toilet lid 206 via a fastening means (e.g., an adhesive, clips, a ring, etc.) and allows users to check and see if they are clean after using the bathroom. More specifically, the toilet lid mirror device 100, with the assistance of the LED light 210, enables a user to easily and clearly view his or her anus, groin and hip areas to make sure that the same have been sufficiently cleaned after urination and/or defecation, and to also apply any medications, ointments or the like to the same.

The size of the toilet lid mirror device 100 can be as per the requirements and needs of the user. The mirror device 100 can be the same shape and size of the toilet seat lid 206, and may have beveled, rounded or flat edges. The mirror 100 is removably attached to the toilet seat lid 206 and can be easily removed while the toilet 202 is being cleaned. The mirror device 100 can also be effectively and easily cleaned with any commonly known cleaning mechanism while the same remains secured to the lid 206. Additionally, the presence of the UV light 212 may also be used to clean and disinfect the various surfaces of the toilet 202 and the device 100.

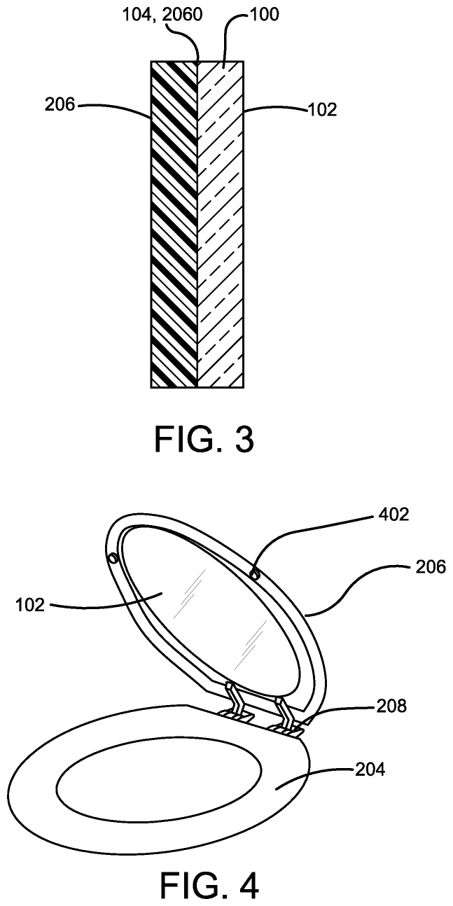

FIG. 3 illustrates a sectional view of one potential embodiment of the toilet lid mirror accessory 100 of the present invention installed along an inner surface 2060 of a toilet lid 206 as per the disclosed architecture. More specifically, the non-reflective surface 104 of the toilet lid mirror 100 adjoins the inner surface 2060 of the toilet lid 206, and the reflective surface 102 of the mirror device 100 is used to maintain cleanliness and personal hygiene. As stated earlier, the mirror 100 can be as big as the toilet seat lid and may have beveled or rounded edges. It should be appreciated that the mirror 100 can be cleaned easily by wiping with toilet paper, or by washing the same with soap and water.

FIG. 4 illustrates a perspective view of one potential embodiment of the toilet lid mirror accessory 100 of the present invention installed along an inner surface 2060 of a toilet lid 206 of a toilet 202 as per the disclosed architecture. As shown, the mirror device 100 is comprised of a reflective surface 102 that is visible to a user and that is attached using an adhesive tape or double-sided tape, or through a frame to the interior surface of the toilet lid 206. A stopper 402 is also attached to the underside surface 2060 of the toilet lid 206 to ensure that when the toilet lid 206 is in the closed position and covering the toilet seat 204, the reflective surface 102 of the mirror 100 does not touch the seat cover 204 and thus remains clean.

The toilet lid mirror 100 can be removed easily from the toilet lid 206 and the adhesive can be used again to re-attach the mirror device 100 to the interior surface 2060 of another or the same toilet lid 206. The mirror device 100 may further comprise a decorative design along the edges to provide an aesthetic appeal to the mirror device 100 and the toilet 202. The toilet lid mirror device 100 can be used with both public and private toilets 202.

Certain terms are used throughout the following description and claims to refer to particular features or components. As one skilled in the art will appreciate, different persons may refer to the same feature or component by different names. This document does not intend to distinguish between components or features that differ in name, but not structure or function. As used herein “toilet lid mirror”, “toilet mirror”, “lid mirror”, and “mirror” are interchangeable and refer to the toilet lid accessory device 100 of the present invention.

Notwithstanding the forgoing, the toilet lid mirror accessory device 100 of the present invention and its various components can be of any suitable size and configuration as is known in the art without affecting the overall concept of the invention, provided that it accomplishes the above stated objectives. One of ordinary skill in the art will appreciate that the size, configuration and material of the toilet lid mirror accessory device 100 and its various components as shown in the FIGS. are for illustrative purposes only, and that many other sizes of the toilet lid mirror accessory device 100 are well within the scope of the present disclosure. Although the dimensions of the toilet lid mirror accessory device 100 and its various components are important design parameters for user convenience, the toilet lid mirror accessory device 100 and its various components may be of any size that ensures optimal performance during use and/or that suits user need and/or preferences.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A toilet accessory device for attachment to an underside of a toilet lid, the toilet accessory device comprising:
a front reflective surface;
a rear surface; and
a fastening means.

2. The toilet accessory device as recited in claim 1, wherein the fastening means is comprised of at least one adhesive member.

3. The toilet accessory device as recited in claim 1 wherein the fastening means is comprised of a plurality of clips.

4. The toilet accessory device as recited in claim 2, wherein the front reflective surface comprises a shape and size that corresponds to a shape and side of the toilet lid.

5. The toilet accessory device as recited in claim 4 further comprising at least one LED light.

6. The toilet accessory device as recited in claim 5 further comprising at least one UV light.

7. The toilet accessory device as recited in claim 6 further comprising at least one sensor.

8. The toilet accessory device as recited in claim 7, wherein the at least one sensor is comprised of at least one or a motion sensor or a light sensor.

9. The toilet accessory device as recited in claim 8 further comprising at least one battery.

10. The toilet accessory device as recited in claim 9, wherein the at least one battery is a rechargeable battery and is in electrical communication with each of the at least one LED light, the at least one UV light and the at least one sensor.

11. The toilet accessory device as recited in claim 10 further comprising a timer.

12. The toilet accessory device as recited in claim 11, wherein the timer is activated when the toilet lid is placed in a closed position.

13. The toilet accessory device as recited in claim 12, wherein the activation of the timer in turn activates the at least one UV light.

14. A toilet accessory system comprising:
a toilet lid hingedly attached to a toilet bowl;
a mirror that is shaped and configured to match a shape and a configuration of the toilet lid; and
a fastening means for attaching the mirror to an underside of the toilet lid.

15. The toilet accessory system as recited in claim 14 further comprising at least one LED light and at least one UV light.

16. The toilet accessory system as recited in claim 15 further comprising at least one sensor and at least one battery, wherein the battery and is in electrical communication with each of the at least one LED light, the at least one UV light and the at least one sensor.

17. The toilet accessory system as recited in claim 16 further comprising a timer.

18. The toilet accessory system as recited in claim 17, wherein the timer is activated when the toilet lid is placed in a closed position.

19. The toilet accessory system as recited in claim 18, wherein the activation of the timer in turn activates the at least one UV light.

20. A toilet accessory system comprising:
an oval shaped toilet lid hingedly attached to a toilet bowl;
an oval shaped mirror that is sized to match a size of the toilet lid;
at least one adhesive strip for attaching the oval shaped mirror to an underside of the oval shaped toilet lid;
a battery;
at least one LED light attached to at least one of the oval shaped mirror or the underside of the oval shaped toilet lid; and
at least one UV light attached to at least one of the oval shaped mirror or the underside of the oval shaped toilet lid, wherein the at least one LED light and the at least one UV light are powered by the battery.
